(19) United States
(12) Patent Application Publication
(10) Pub. No.: US 2025/0242508 A1
(43) Pub. Date: Jul. 31, 2025

(54) FOLDING KNIFE

(71) Applicant: Hogue Tool & Machine, Inc., Paso Robles, CA (US)

(72) Inventor: James David Bruhns, Templeton, CA (US)

(73) Assignee: Hogue Tool & Machine, Inc., Paso Robles, CA (US)

(21) Appl. No.: 19/024,062

(22) Filed: Jan. 16, 2025

Related U.S. Application Data
(60) Provisional application No. 63/627,139, filed on Jan. 31, 2024.

(51) Int. Cl. B26B 5/00

(52) U.S. Cl. CPC B26B 5/00

(57) ABSTRACT

Folding knives have an elongated frame, a blade holder pivotally connected to the elongated frame to move between a closed position and an open position, the blade holder having a blade mount facility configured to removably receive a blade, and an ejector movably connected to the blade holder and operable to detach the blade when the blade is received on the blade mount facility. The blade holder may be operable to move to an intermediate position between the open and closed position, and the ejector may be operable to detach a blade only in the intermediate position. The ejector may be constrained to render the ejector inoperable to detach a blade in the open position. The ejector may be constrained to render the ejector inoperable to detach a blade in the closed position. The ejector may be an elongated bar.

### CROSS-REFERENCE TO RELATED APPLICATION

This application claims the benefit of U.S. Provisional Patent Application No. 63/627,139 filed on Jan. 31, 2024, entitled “FOLDING KNIFE WITH INTEGRAL BLADE EJECTOR,” which is hereby incorporated by reference in its entirety for all that is taught and disclosed therein.

### FIELD OF THE INVENTION

The present invention relates to knives, and more particularly to a folding knife that restrains the blade in use while also reliably ejecting the blade in a safe manner.

### BACKGROUND AND SUMMARY OF THE INVENTION

Hunting knives are a common tool for the outdoorsman. They are considered one of the most important items to be carried during any wilderness activity. To ensure ease of carry, the hunting knife should be as light as possible and no larger than is necessary to perform an intended task. For example, a hunting knife used for field dressing a deer, wild pig, or other animal to harvest meat must be sufficiently large while remaining light. The hunting knife must also remain sharp throughout the field dressing activity until harvesting is complete. This is where the size of knife and other factors, such as type of knife steel, contribute to the user's knife selection while pre-planning for these field activities.

A common problem occurs when, for a variety of reasons, the selected knife does not stay sharp even after thoughtful planning has occurred. Over the past several years, a solution to this problem has evolved where a knife handle that allows for a replaceable sharp blade has gained universal acceptance and popularity. Some common makers of these knives with replaceable blades are: Havalon Knives of Cincinnati, OH, Gerber Gear of Portland, OR, and Kershaw Knives of Tualatin, OR. Many of these relatively new knife products have been designed poorly with respect to overall size and weight, but have succeeded with high praise for their ability to provide a very inexpensive replacement blade in the field to assure a sharp edge regardless of extended heavy cutting activities. The user can replace a dull blade with a new sharp blade as needed until field activities are completed.

While many knife manufacturing companies have acknowledged this trend for products with replaceable blades, Havalon Knives has emerged as the clear leader in this product category. In fact, the word Havalon has become synonymous among users and retailers for this product almost universally. Another contributing element to this universally acknowledged term (Havalon), is that other knife makers have found it more convenient and economical to design and produce products using blades provided by Havalon Knives. These blades can be purchased from Havalon Knives in bulk volume very inexpensively, work very well, and are a proven product that the end user wants and recognizes.

However, some problems with replaceable blade knives have been identified by end users during normal use of these knives. The most concerning is when the replaceable blade becomes unrestrained and slips out of the handle. This happens when the user is manipulating the knife with enough force to bend the rear of the blade high enough to achieve the proper elevation for removal of the blade. When this condition exists, a simple push of the blade or pull of the handle will cause the blade to disengage from the handle, often putting the user in danger of encountering a sharp edge and exposing them to cuts or potentially more serious injury. A second problem occurs when the user has decided to replace a blade under field or other use conditions where their hands might be slippery because they are bloody or greasy. When this condition exists, it is difficult to lift the rear of the blade and push the lifted blade forward far enough to release the blade from the handle. This activity often results in the hand slipping from the grip of the blade and exposing the hand or fingers to the sharp edge of the blade and again exposing the user to potential injury. One approach that mitigates this hand slipping hazard is disclosed by US Patent Publication No. 2023/0381984 A1 to Bruhns, which is hereby incorporated by reference for all that is taught therein. While other hazards exist, they are more or less similar to virtually all other types of knives.

Therefore, a need exists for a new and improved folding knife that restrains the blade in use while also reliably ejecting the blade in a safe manner. In this regard, the various embodiments of the present invention substantially fulfill at least some of these needs. In this respect, the folding knife according to the present invention substantially departs from the conventional concepts and designs of the prior art, and in doing so provides an apparatus primarily developed for the purpose of restraining the blade in use while also reliably ejecting the blade in a safe manner.

The present invention provides an improved folding knife, and overcomes the above-mentioned disadvantages and drawbacks of the prior art. As such, the general purpose of the present invention, which will be described subsequently in greater detail, is to provide an improved folding knife that has all the advantages of the prior art mentioned above.

To attain this, the preferred embodiment of the present invention essentially comprises an elongated frame, a blade holder pivotally connected to the elongated frame to move between a closed position and an open position, the blade holder having a blade mount facility configured to removably receive a blade, and an ejector movably connected to the blade holder and operable to detach the blade when the blade is received on the blade mount facility. The blade holder may be operable to move to an intermediate position between the open and closed position, and the ejector may be operable to detach a blade only in the intermediate position. The ejector may be constrained to render the ejector inoperable to detach a blade in the open position. The ejector may be constrained to render the ejector inoperable to detach a blade in the closed position. The ejector may be an elongated bar. There are, of course, additional features of the invention that will be described hereinafter and which will form the subject matter of the claims attached.

There has thus been outlined, rather broadly, the more important features of the invention in order that the detailed description thereof that follows may be better understood and in order that the present contribution to the art may be better appreciated.

### BRIEF DESCRIPTION OF THE DRAWINGS

FIG. 1 is an isometric view of the current embodiment of a folding knife constructed in accordance with the principles of the present invention with the blade holder in the open position.

FIG. 2 is an exploded view of the folding knife of FIG. 1 from the left side.

FIG. 3 is an exploded view of the folding knife of FIG. 1 from the right side.

FIG. 4 is a right side view of the folding knife of FIG. 1 with the right handle removed and the blade holder in the closed position.

FIG. 5 is a right side view of the folding knife of FIG. 1 with the right handle removed and the blade holder in the intermediate position.

FIG. 6 is a front view of the folding knife of FIG. 1 with the left handle removed and the blade holder in the intermediate position.

FIG. 7 is an enlarged partial front view of the folding knife of FIG. 1 with the blade holder in the intermediate position.

FIG. 8 is a right side view of the folding knife of FIG. 1 with the right handle removed, the blade holder in the intermediate position, and the blade ejected.

FIG. 9 is a front view of the folding knife of FIG. 1 with the right handle removed, the blade holder in the intermediate position, and the blade ejected.

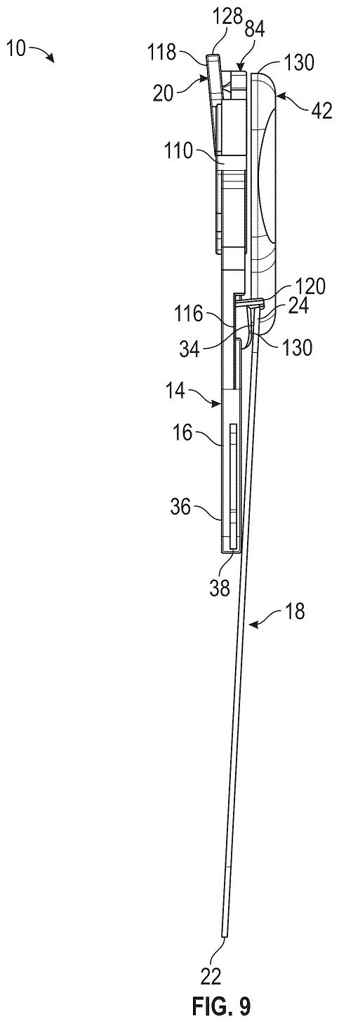

FIG. 10 is a right side view of the folding knife of FIG. 1 with the right handle removed and the blade holder in the open position.

FIG. 11 is a top view of the folding knife of FIG. 1 with the left handle removed and the blade holder in the open position.

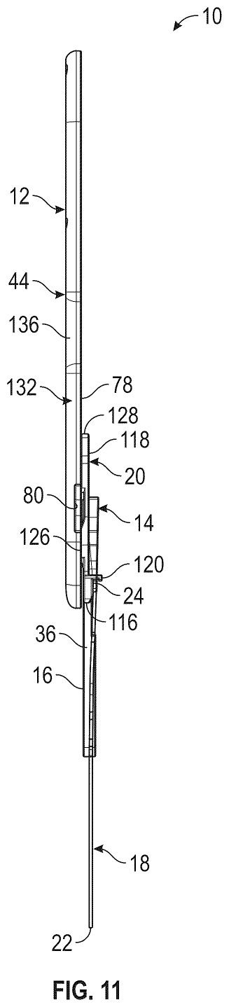

FIG. 12 is a sectional view taken along the ejector of the folding knife of FIG. 1 with the blade holder in the partially open position.

The same reference numerals refer to the same parts throughout the various figures.

### DESCRIPTION OF THE CURRENT EMBODIMENT

An embodiment of the folding knife of the present invention is shown and generally designated by the reference numeral 10.

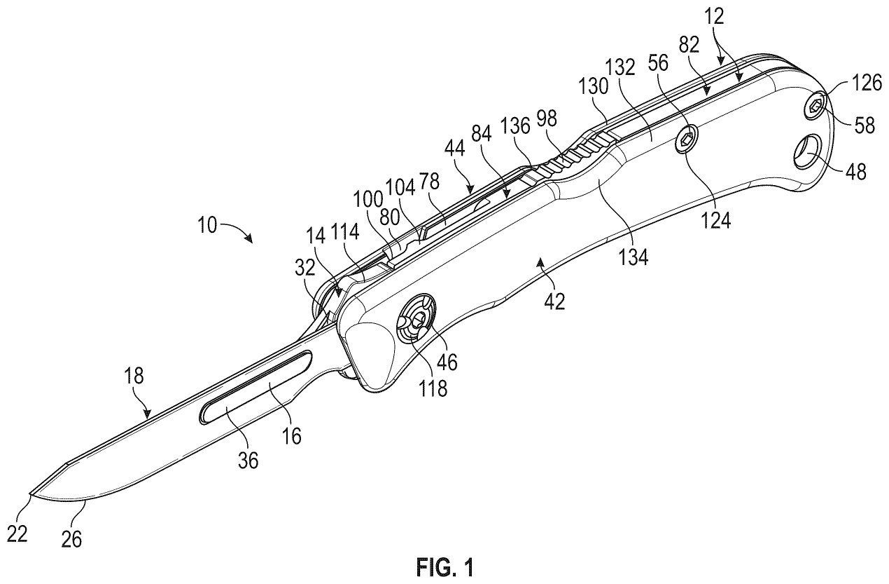

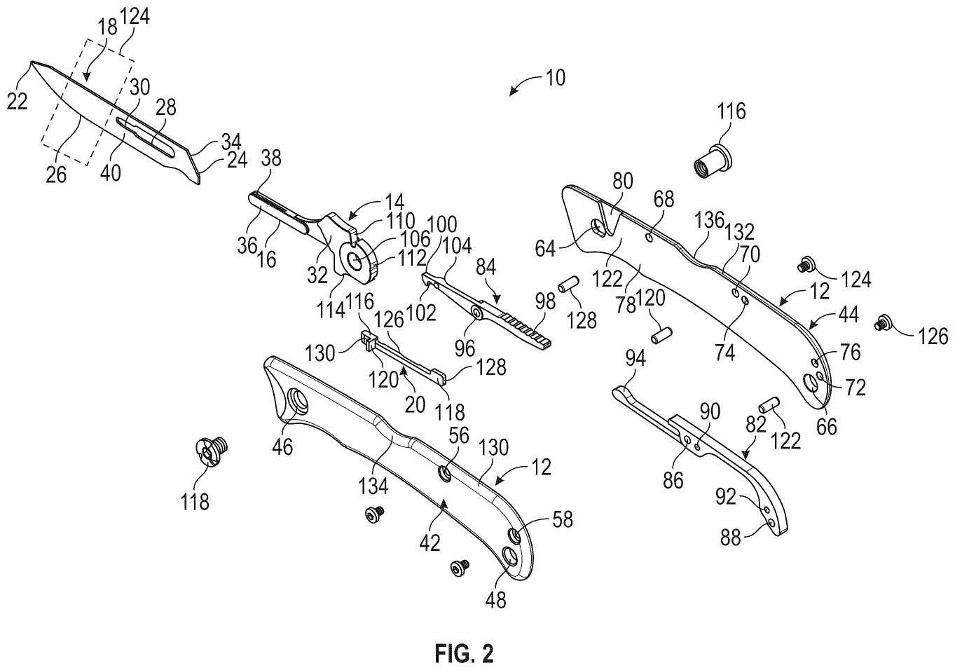

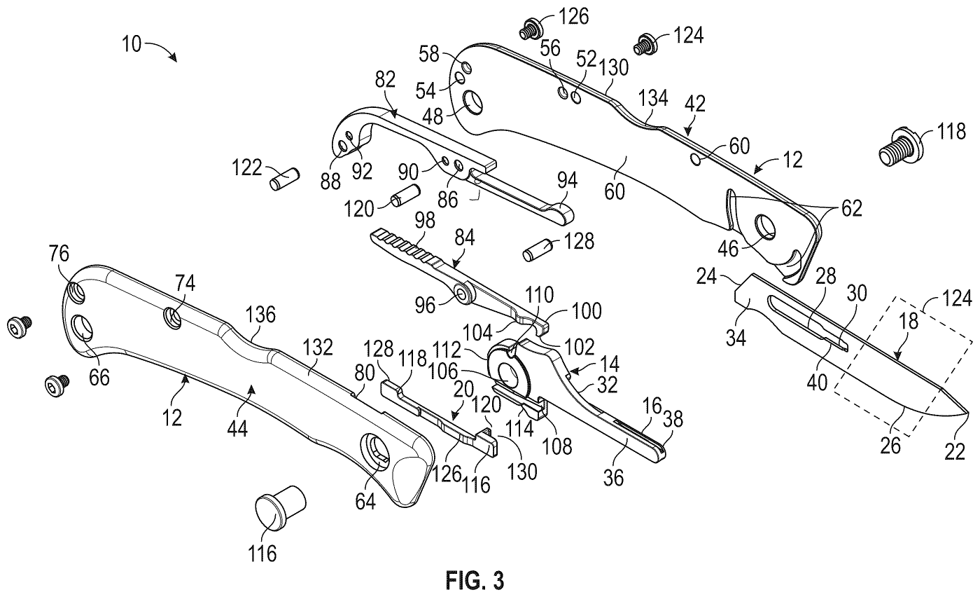

FIGS. 1-3 illustrate the improved folding knife 10 of the present invention. More particularly, the folding knife has an elongated frame 12 and a blade holder 14 pivotally connected to the elongated frame to move between a closed position and an open position. The blade holder has a blade mount facility 16 configured to removably receive a blade 18, which is flexible in the current embodiment. An ejector 20 is movably connected to the blade holder and operable to detach the blade when the blade is received on the blade mount facility. The ejector is an elongated bar in the current embodiment.

The blade 18 has a forward end 22 and an opposed rear end 24, a sharp edge 26, and defines an elongated aperture 28 having a constriction at one end 30. The blade attachment facility 16 has a blade support surface 32 configured to abut a major face portion 34 of the blade proximate the elongated aperture. An elongated boss 36 is sized to closely receive the elongated aperture of the blade. The elongated boss defines an undercut 38 configured to receive a blade portion 40 at the constriction to prevent separation of the blade from the blade support surface when the planar blade is in an installed condition with the elongated boss received in the elongated aperture. The elongated boss is a feature of the blade holder that is a standard elongated grooved pedestal configured to receive standard scalpel blades having elongated apertures receiving the standard elongated grooved pedestal.

The elongated frame 12 includes a left handle 42 and a right handle 44. The left handle defines a front aperture 46, a rear lanyard aperture 48, three dowel apertures 50, 52, 54, and two screw apertures 56, 58. The left handle has an interior surface 60 that defines a semicircular groove 62 partially encircling the front aperture 46. The right handle defines a front aperture 64, rear lanyard aperture 66, three dowel apertures 68, 70, 72, and two screw apertures 74, 76. The right handle has an interior surface 78 that defines a V-shaped recess 80 adjacent to the front aperture 64.

A back spacer 82 and a movable lock element 84 are secured between the left handle 42 and the right handle 44 when the folding knife 10 is assembled. The back spacer defines two dowel apertures 86, 88, two screw apertures 90, 92, and a forward spring portion 94. The movable lock element defines a dowel aperture 96 and includes a ridged rear end 98 and a forward end 100. The forward end includes a downward-facing hook 102 and a right-facing protrusion 104.

The blade holder 14 defines a rear aperture 106 and an elongated channel 108 located adjacent to the rear aperture. The blade holder also defines an open position notch 110, and an intermediate position detent 112 located 90° from the open position notch, and a closed position detent 114 located 90° from the intermediate position detent.

When the folding knife 10 is assembled, the elongated channel 108 defined by the blade holder 14 receives the ejector 20. The front aperture 46 of the left handle 42, rear aperture 106 of the blade holder, and the front aperture 64 of the right handle 44 are axially registered such that a pivot pin 116 can be received by the front aperture 46, rear aperture 106, and front aperture 64. The pivot pin is secured in place by a pivot screw 118. The back spacer 82 is positioned between the left and right handle such that the two dowel apertures 86, 88 defined by the back spacer are axially registered with the dowel apertures 52, 54 defined by the left handle and the dowel apertures 70, 72 defined by the right handle such that dowel pins 120, 122 can be received by the dowel apertures 52, 54, 70, 72, 86, 88. The back spacer is positioned between the left and right handle such that the two screw apertures 90, 92 are axially registered with the two screw apertures 56, 58 defined by the left handle and the two screw apertures 74, 76 defined by the right handle such that screws 124, 126 can be threadedly received by the screw apertures 56, 58, 74, 76, 90, 92. The movable lock element 84 is positioned between the left and right handle such that the dowel aperture 96 is axially registered with dowel aperture 50 defined by the left handle and dowel aperture 68 defined by the right handle such that dowel pin 128 can be received by the dowel apertures 50, 68, 96 to enable the movable lock element to pivot about dowel pin 128.

When the folding knife 10 is assembled, the top 130 of the left handle 42 and the top 132 of the right handle 44 each define a notch 134, 136 that expose the ridged rear end 98 of the movable lock element 84. The forward spring portion 94 of the back spacer 82 is beneath the ridged rear end of the movable lock element and biases the ridged rear end of the movable lock element upwards. This biasing action pushes the downward-facing hook 102 on the movable lock element downwards against the blade holder such that the downward-facing hook encounters the open position notch 110, intermediate position detent 112, and closed position detent 114 as the blade holder pivots between the closed position and the open position.

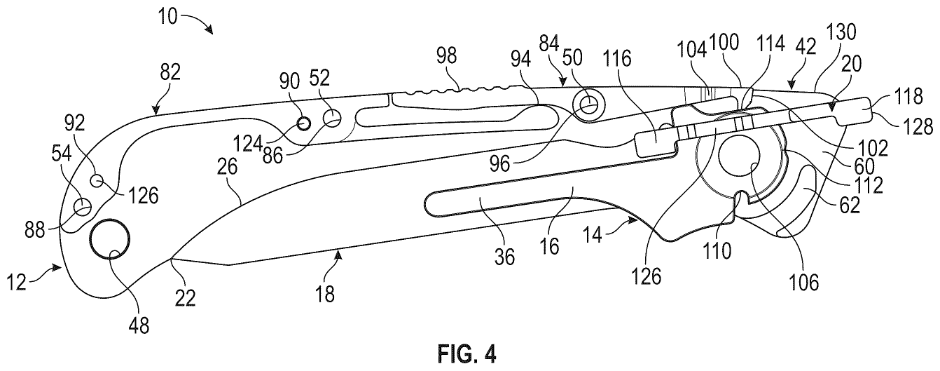

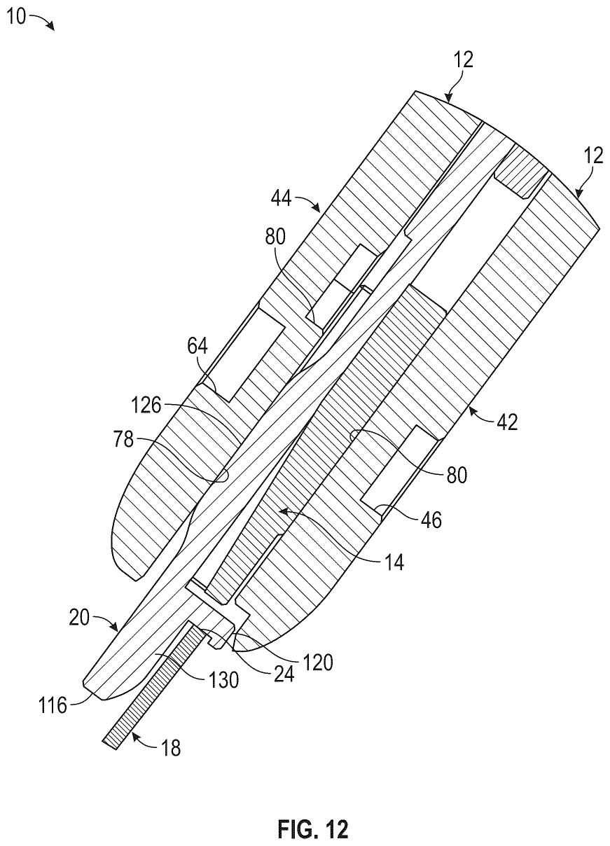

FIGS. 4 & 12 illustrate the improved folding knife 10 of the present invention. More particularly, the folding knife is shown with the blade holder 14 in the closed position in FIG. 4, and the folding knife is shown with the blade holder in a partially open position in FIG. 12. The ejector 20 is received in the elongated channel 108 defined by the blade holder. The ejector is captured between the blade holder and the interior surface 78 of the right handle 44 to render the ejector inoperable to detach the blade 18 when the folding knife 10 is in the closed position. Specifically, the right handle of the elongated frame 12 has an ejector limit surface 122 surrounding the V-shaped recess 80 preventing operation of the ejector to detach the blade when the blade holder is in the closed position. The ejector has a first end 116 extending toward the blade mount facility 16 and configured to contact and motivate the blade, and an opposed second end 118. The second end is a portion of the ejector that protrudes from the elongated frame when the blade holder is in the closed position, and the ejector is rotationally engaged to the blade holder to motivate movement of the blade holder toward the open position in response to force on the ejector. The first end includes a leftward-facing protrusion 120. The leftward facing protrusion is captured between the rear end 24 of the blade and the blade holder to limit travel of the ejector away from the rear end of the blade. Pressure exerted by the rear end of the blade against the first end of the ejector biases the ejector toward a neutral position where the ejector does not tilt. The blade defines a major plane 124, and the ejector is operable to tilt between a first position parallel to the major plane, which the ejector occupies when the blade holder is in the open and closed position, and a second tilted position angularly offset from the major plane that the ejector occupies when the blade holder is in the intermediate position. The downward-facing hook 102 is received within the closed position detent 114 to releasably secure the blade holder in the closed position.

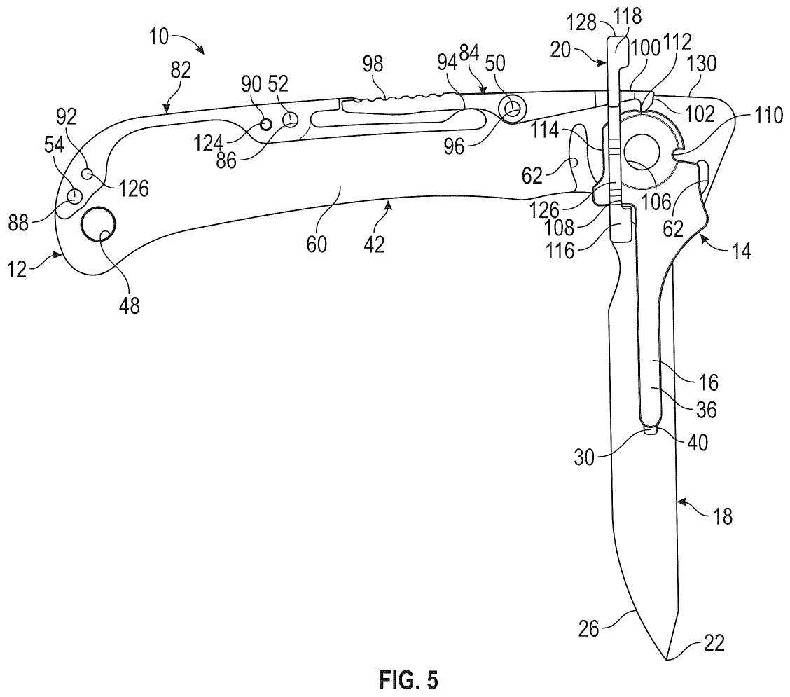

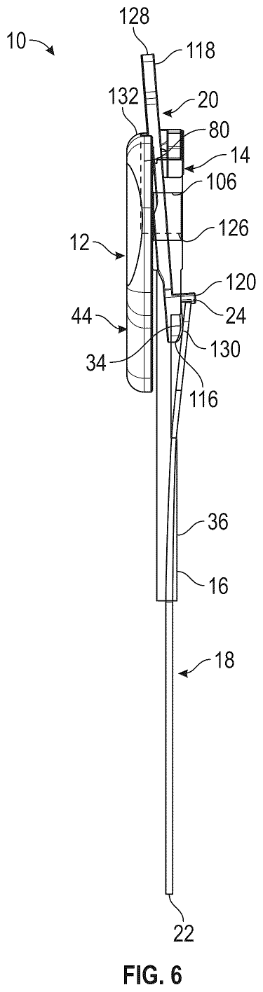

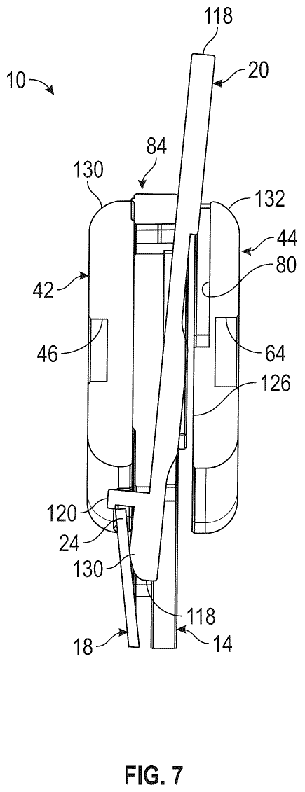

FIGS. 5-7 illustrate the improved folding knife 10 of the present invention. More particularly, the folding knife is shown with the blade holder 14 in the intermediate position. The ejector 20 is received in the elongated channel 108 defined by the blade holder. The downward-facing hook 102 is received within the intermediate position detent 112 to releasably secure the blade holder in the intermediate position and to provide a tactile indication to the user that the blade 18 is capable of being ejected. The V-shaped recess 80 defined by the right handle 44 of the elongated frame 12 is configured to receive a protruding portion 126 of the ejector to enable the ejector to detach the blade when the blade holder is in an intermediate position between the open and closed positions. The V-shaped recess is positioned to enable the ejector to detach the blade when the blade holder is 90° from the closed position. The ejector is operable to eject the blade only when the ejector is in the tilted position. The V-shaped recess defined by the right handle of the elongated frame is configured to prevent tilting of the ejector except when the blade holder is in a limited range of positions within the range of motion of the blade holder. The movable lock element 84 is a selected frame portion on the elongated frame that includes a right-facing protrusion 104 configured to cause tilting of the ejector in the limited range of positions. The ejector is a unitary element having a first surface 128 configured for direct force by a finger and a second surface 130 directly contacting the blade to motivate ejection of the blade by lifting the rear of the blade, followed by the leftward-facing protrusion 120 pushing forward on the blade.

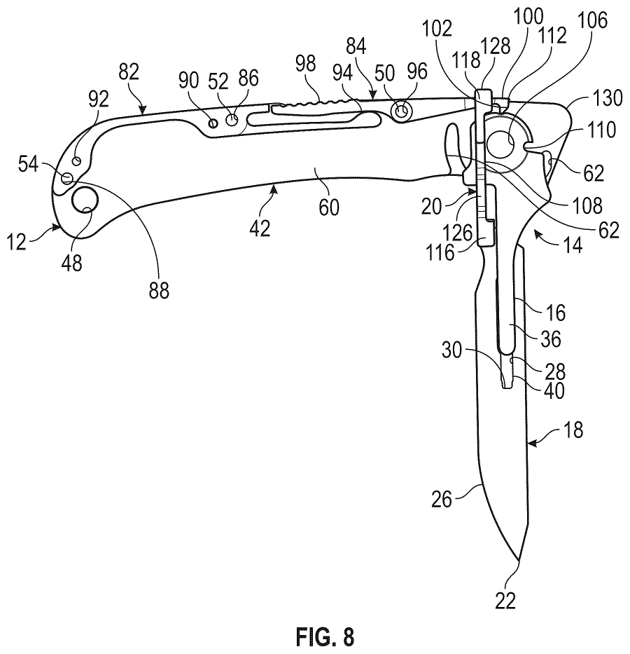

FIGS. 8 & 9 illustrate the improved folding knife 10 of the present invention. More particularly, the process of detaching the blade 18 from the blade holder 14 is shown. When the blade holder is in the intermediate position, the rear 24 of the blade is lifted by the second surface 130 as a result of the tilt of the ejector 20 so the rear of the blade is above the elongated boss 36. The user can now push forward on the ejector to push the blade free of the undercut 38 using the leftward-facing protrusion 120 on the ejector and safely remove the blade from the blade holder without contacting the blade with the hand or fingers. The blade can be ejected into a convenient receptacle or other appropriate container so the used blade is never directly handled. It should be appreciated that the rear of the blade is allowed to snap down and around the rear portion of the elongated boss only when the blade holder is rotated out of the intermediate position in either direction towards the open or closed position. Sufficient friction forces exist between the blade and the elongated boss to prevent the blade from unintentionally detaching from the elongated boss while the blade holder is in the intermediate position unless the user pushes on first surface 128 of the ejector.

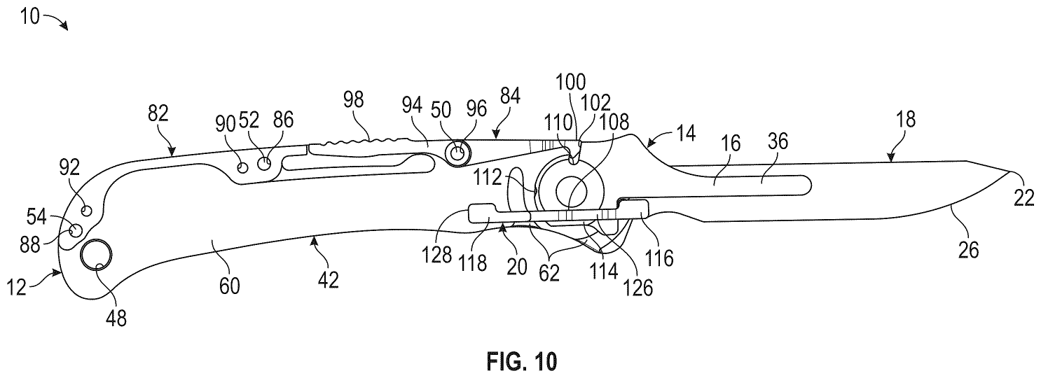

FIGS. 10 & 11 illustrate the improved folding knife 10 of the present invention. More particularly, the folding knife is shown with the blade holder 14 in the open position. The ejector 20 is received in the elongated channel 108 defined by the blade holder. The ejector is captured between the blade holder and the right handle 44 to render the ejector inoperable to detach the blade 18 when the folding knife 10 is in the open position. Specifically, the right handle of the elongated frame 12 has an ejector limit surface 122 surrounding the V-shaped recess 80 preventing operation of the ejector to detach the blade when the blade holder is in the closed position. Pressure exerted by the rear end 24 of the blade against the first end 116 of the ejector biases the ejector toward a neutral position where the ejector does not tilt. The downward-facing hook 102 of the movable lock element 84 is received within the open position notch 110 to releasably secure the blade holder in the open position in the style of a lock back knife. The forward spring portion 94 of the back spacer 82 is beneath the ridged rear end of the movable lock element and biases the ridged rear end of the movable lock element upwards to urge the downward-facing hook into the open position notch. Only when the user applies sufficient downward pressure to the ridged rear end 98 of the movable lock element to overcome the force of the forward spring portion of the back spacer and lift the downward-facing hook out of the open position notch can the blade holder be rotated towards the closed position.

While a current embodiment of a folding knife has been described in detail, it should be apparent that modifications and variations thereto are possible, all of which fall within the true spirit and scope of the invention. For example, although a lock back style blade lock has been disclosed to hold the blade older in the open position, other locking mechanisms could be used including liner lock and button lock style locking mechanisms. Furthermore, the second end being a portion of the ejector that protrudes from the elongated frame when the blade holder is in the closed position could be omitted and replaced by the user gripping the blade to open the folding knife. In addition, the right-facing protrusion on the movable lock element could be omitted and replaced by the user lifting the rear of the blade with a fingernail above the elongated boss prior to using the ejector to push off the blade. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A folding knife comprising:
an elongated frame;
a blade holder pivotally connected to the elongated frame to move between a closed position and an open position;
the blade holder having a blade mount facility configured to removably receive a blade; and
an ejector movably connected to the blade holder and operable to detach the blade when the blade is received on the blade mount facility.

2. The folding knife of claim 1 wherein the blade holder is operable to move to an intermediate position between the open and closed position, and wherein the ejector is operable to detach a blade only in the intermediate position.

3. The folding knife of claim 1 wherein the ejector is constrained to render the ejector inoperable to detach a blade in the open position.

4. The folding knife of claim 1 wherein the ejector is constrained to render the ejector inoperable to detach a blade in the closed position.

5. The folding knife of claim 1 wherein the ejector is an elongated bar.

6. The folding knife of claim 5 wherein the ejector has a first end extending toward the blade mount facility and configured to contact and motivate a blade, and an opposed second end.

7. The folding knife of claim 5 wherein the second end extends from the elongated frame when the blade holder is in the closed position.

8. The folding knife of claim 1 wherein the blade holder defines an elongated channel receiving the ejector.

9. The folding knife of claim 1 wherein the elongated frame has an ejector limit surface preventing operation of the ejector to detach the blade when the blade holder is in the open position.

10. The folding knife of claim 1 wherein the elongated frame has an ejector limit surface preventing operation of the ejector to detach the blade when the blade holder is in the closed position.

11. The folding knife of claim 1 wherein the elongated frame defines a recess configured to receive a portion of the ejector to enable the ejector to detach the blade when the blade holder is in an intermediate position between the open and closed positions.

12. The folding knife of claim 11 wherein the recess is positioned to enable the ejector to detach the blade when the blade holder is 90° from the closed position.

13. The folding knife of claim 1 wherein a portion of the ejector protrudes from the elongated frame when the blade holder is in the closed position, and the ejector is rotationally engaged to the blade holder to motivate movement of the blade holder toward the open position in response to force on the ejector.

14. The folding knife of claim 1 wherein the blade defines a major plane and wherein the ejector is operable to tilt between a first position parallel to the major plane and a second tilted position angularly offset from the major plane.

15. The folding knife of claim 14 wherein the ejector is operable to eject a blade only when the ejector is in the tilted position.

16. The folding knife of claim 14 wherein the elongated frame is configured to prevent tilting of the ejector except when the blade holder is in a limited range of positions within the range of motion of the blade holder.

17. The folding knife of claim 14 wherein a selected frame portion includes a protrusion configured to cause tilting of the ejector in the limited range of positions.

18. The folding knife of claim 17 wherein the selected frame portion is a movable lock element on the elongated frame.

19. The folding knife of claim 1 wherein the ejector is a unitary element having a first surface configured for direct force by a finger and a second surface directly contacting the blade to motivate ejection of the blade.

20. The folding knife of claim 1 wherein the blade holder includes a standard elongated grooved pedestal configured to receive standard scalpel blades having elongated apertures receiving the standard elongated grooved pedestal.
